(19) United States
(12) Patent Application Publication
(10) Pub. No.: US 2023/0198274 A1
(43) Pub. Date: Jun. 22, 2023

(54) SYSTEMS AND METHODS FOR ESTIMATING POWER SOURCE CAPACITY OF AN IMPLANTABLE CONTROL MODULE OF AN ELECTRICAL STIMULATION SYSTEM

(71) Applicant: Boston Scientific Neuromodulation Corporation, Valencia, CA (US)

(72) Inventors: Farrokh Aghaeepour, Valencia, CA (US); Robert Graham Lamont, Van Nuys, CA (US); Kenneth Tsang, Los Angeles, CA (US)

(21) Appl. No.: 18/084,282

(22) Filed: Dec. 19, 2022

Related U.S. Application Data
(60) Provisional application No. 63/292,656, filed on Dec. 22, 2021.

(51) Int. Cl. H02J 7/00; A61N 1/378; A61N 1/36

(52) U.S. Cl. CPC H02J 7/0048; A61N 1/378; A61N 1/3614

(57) ABSTRACT

An electrical stimulation system includes a lead having electrodes disposed along a distal portion of the lead; and an implantable control module coupled, or coupleable, to the lead and configured for implantation in a patient. The implantable control module includes a power source, and a processor coupled to the power source and configured for directing electrical stimulation through the electrodes of the lead using the power source and for calculating an estimate of a capacity or energy of the power source that has been used based, at least in part, on the directed electrical stimulation.

### CROSS-REFERENCE TO RELATED APPLICATIONS

This application claims the benefit under 35 U.S.C. § 119(e) of U.S. Provisional Patent Application Ser. No. 63/292,656, filed Dec. 22, 2021, which is incorporated herein by reference.

### FIELD

The present invention is directed to the area of implantable electrical stimulation systems and methods of making and using the systems. The present invention is also directed to systems and methods for estimating power source capacity of an implantable control module of an electrical stimulation system, as well as methods of making and using the implantable control modules and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Deep brain stimulation can be used to treat a variety of diseases and disorders.

Stimulators have been developed to provide therapy for a variety of treatments. A stimulator can include a control module (with a pulse generator) and one or more stimulator electrodes. The one or more stimulator electrodes can be disposed along one or more leads, or along the control module, or both. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is an electrical stimulation system that includes a lead having electrodes disposed along a distal portion of the lead; and an implantable control module coupled, or coupleable, to the lead and configured for implantation in a patient. The implantable control module includes a power source, and a processor coupled to the power source and configured for directing electrical stimulation through the electrodes of the lead using the power source and for calculating an estimate of a capacity or energy of the power source that has been used based, at least in part, on the directed electrical stimulation.

In at least some aspects, the estimate of the capacity or energy of the power source that has been used includes an estimate of the charge or power used during each electrical stimulation instance in which the processor directs the electrical stimulation through the electrodes of the lead. In at least some aspects, the estimate of the charge used during an electrical stimulation instance includes a summation of, for each of the electrodes used for delivery of the electrical stimulation during the electrical stimulation instance, a) a product of a stimulation amplitude, pulse width, pulse frequency, and duration of the electrical stimulation instance for the electrode or b) a product of the stimulation amplitude, pulse width, and pulse frequency integrated over the duration of the electrical stimulation instance for the electrode. In at least some aspects, the estimate of the power used during an electrical stimulation instance includes a summation of, for each of the electrodes used for delivery of the electrical stimulation during the electrical stimulation instance, a) a product of a compliance voltage, stimulation amplitude, pulse width, pulse frequency, and duration of the electrical stimulation instance for the electrode or b) a product of the compliance voltage, stimulation amplitude, pulse width, and pulse frequency integrated over the duration of the electrical stimulation instance for the electrode. In at least some aspects, the compliance voltage is determined using an estimate or measurement of tissue impedance and the stimulation amplitude.

In at least some aspects, the estimate of the capacity or energy of the power source that has been used is also based on an estimate of charge or power used for one or more other operations of the implantable pulse generator. In at least some aspects, the estimate of the charge or power used for the one or more other operations of the implantable pulse generator includes at least one calculation or estimation of the charge or power used for at least one instance of at least one of the one or more other operations. In at least some aspects, the estimate of the charge or power used for the one or more operations of the implantable pulse generator uses an overhead consumption value for accounting for at least one of the one or more other operations. In at least some aspects, the estimate of the charge or power used for the one or more operations of the implantable pulse generator uses an average charge consumption over a predefined period of time for at least one of the one or more other operations. In at least some aspects, the one or more other operations of the implantable pulse generator include at least one of the following: operation of the processor; operation of digital timers; operation of a step-up converter; operation of current sources; operation of reference sources; operation of a memory; or operation of communications components of the implantable pulse generator.

Another aspect is a method for estimating a capacity or energy of a power source of an implantable control module of an electrical stimulation system that has been used during electrical stimulation. The method includes estimating a charge or power used during each of a plurality of electrical stimulation instances; combining the estimates of the charge or power used during each of the electrical stimulation instances; determining the capacity or energy of the power source that has been used utilizing the combined estimates of the charge or power used during each of the electrical stimulation instances; and, optionally, providing a warning to a patient or device when the determined capacity or energy exceeds a threshold value.

In at least some aspects, estimating the charge used during an electrical stimulation instance includes determining a summation of, for each of the electrodes used for delivery of the electrical stimulation during the electrical stimulation instance, a) a product of a stimulation amplitude, pulse width, pulse frequency, and duration of the electrical stimulation instance for the electrode orb) a product of the stimulation amplitude, pulse width, and pulse frequency integrated over the duration of the electrical stimulation instance for the electrode. In at least some aspects, estimating the power used during an electrical stimulation instance includes determining a summation of, for each of the electrodes used for delivery of the electrical stimulation during the electrical stimulation instance, a) a product of a compliance voltage, stimulation amplitude, pulse width, pulse frequency, and duration of the electrical stimulation instance for the electrode or b) a product of the compliance voltage, stimulation amplitude, pulse width, and pulse frequency integrated over the duration of the electrical stimulation instance for the electrode. In at least some aspects, the method further includes determining the compliance voltage using an estimate or measurement of tissue impedance and the stimulation amplitude.

In at least some aspects, the method further includes estimating a charge or power used for one or more other operations of the implantable pulse generator; and combining the estimates of the charge or power used for the one or more other operations, wherein determining the capacity or energy of the power source that has been used includes determining the capacity or energy of the power source that has been used utilizing the combined estimates of the charge or power used during each of the electrical stimulation instances and the combined estimate of the charge or power used for the one or more other operations. In at least some aspects, estimating the charge or power used for the one or more other operations of the implantable pulse generator includes calculating or estimating the charge or power used for at least one instance of at least one of the one or more other operations. In at least some aspects, estimating the charge or power used for the one or more operations of the implantable pulse generator includes using an overhead consumption value for accounting for at least one of the one or more other operations. In at least some aspects, estimating the charge or power used for the one or more operations of the implantable pulse generator includes using an average charge consumption over a predefined period of time for at least one of the one or more other operations. In at least some aspects, the one or more other operations of the implantable pulse generator include at least one of the following: operation of the processor; operation of digital timers; operation of a step-up converter; operation of current sources; operation of reference sources; operation of a memory; or operation of communications components of the implantable pulse generator.

In at least some aspects, the method further includes delivering electrical stimulation to a patient for each of the electrical stimulation instances.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is a schematic overview of one embodiment of components of a stimulation system, including an electronic subassembly disposed within a control module; and

FIG. 4 is a flowchart of a method for estimating a capacity of a power source that has been used.

### DETAILED DESCRIPTION

The present invention is directed to the area of implantable electrical stimulation systems and methods of making and using the systems. The present invention is also directed to systems and methods for estimating power source capacity of an implantable control module of an electrical stimulation system, as well as methods of making and using the implantable control modules and electrical stimulation systems.

Suitable implantable electrical stimulation systems include, but are not limited to, a least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated herein by reference. In the discussion below, a percutaneous lead will be exemplified, but it will be understood that the methods and systems described herein are also applicable to paddle leads and other leads.

A percutaneous lead for electrical stimulation (for example, deep brain, spinal cord, or peripheral nerve stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the systems and leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

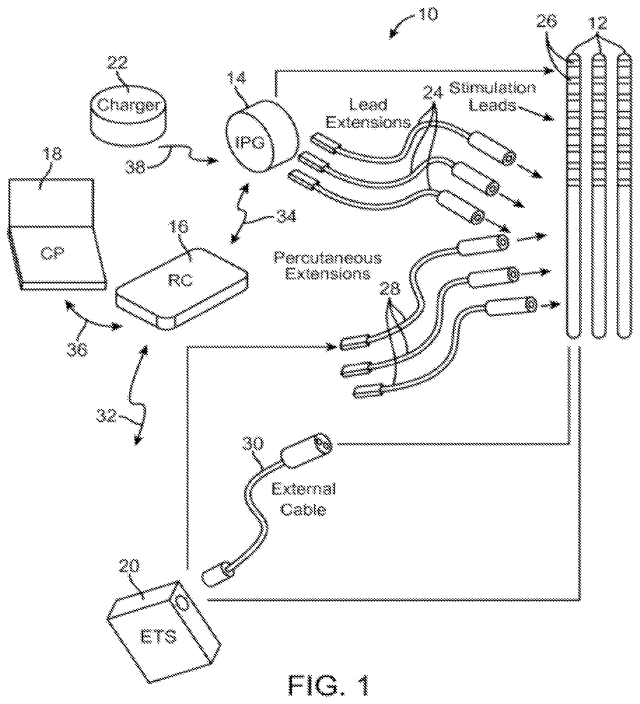

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity or at any other suitable site. The implantable pulse generator can have multiple stimulation channels which may be independently programmable to control the magnitude of the current stimulus from each channel. In some embodiments, the implantable pulse generator can have any suitable number of stimulation channels including, but not limited to, 4, 6, 8, 12, 16, 32, or more stimulation channels. The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14. In at least some embodiments, the RC 16 can be a mobile phone, tablet, desktop computer, or the like.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is incorporated herein by reference in its entirety. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated herein by reference in their entireties.

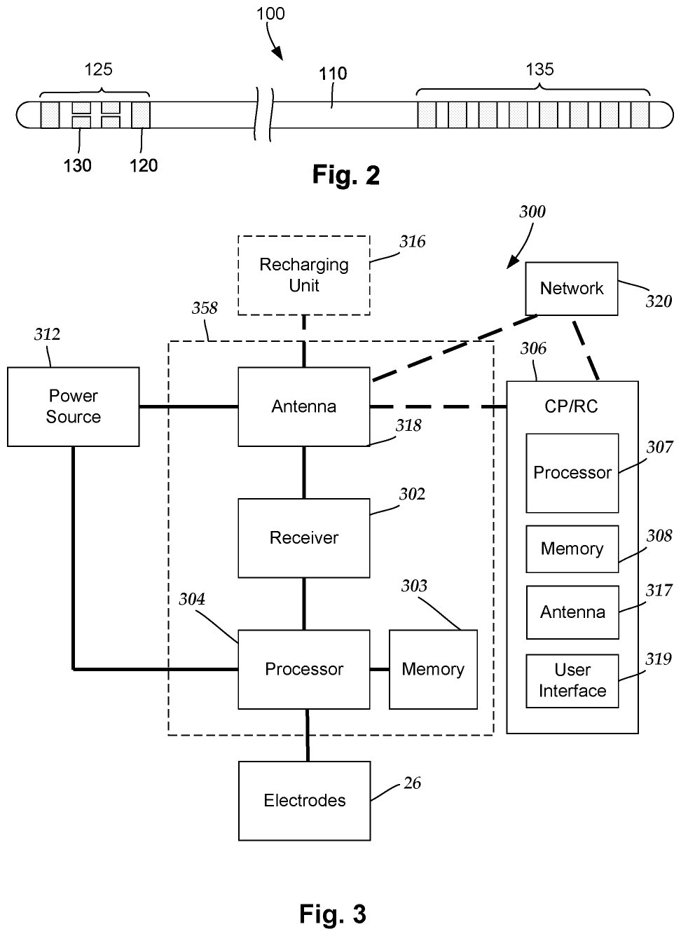

FIG. 2 illustrates one embodiment of a lead 100 with electrodes 125 disposed at least partially about a circumference of the lead 100 along a distal end portion of the lead 100 and terminals 135 disposed along a proximal end portion of the lead 100. The lead 100 can be implanted near or within the desired portion of the body to be stimulated such as, for example, the brain, spinal cord, or other body organs or tissues. In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 100 can be inserted into the cranium and brain tissue with the assistance of a stylet (not shown). The lead 100 can be guided to the target location within the brain using, for example, a stereotactic frame and a microdrive motor system. In at least some embodiments, the microdrive motor system can be fully or partially automatic. The microdrive motor system may be configured to perform at least one of the following actions (alone or in combination): insert the lead 100, advance the lead 100, retract the lead 100, or rotate the lead 100.

In at least some embodiments, measurement devices coupled to the muscles or other tissues affected by the target neurons or neural structures, or a unit responsive to the patient or clinician, can be coupled to the IPG 14 or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 100 for deep brain stimulation can include stimulation electrodes, recording electrodes, or both. In at least some embodiments, the lead 100 is rotatable so that the stimulation electrodes can be aligned with the target neurons after the neurons have been located using the recording electrodes.

Stimulation electrodes may be disposed on the circumference of the lead 100 to stimulate the target neurons. Stimulation electrodes may be ring shaped so that current projects from each electrode radially from the position of the electrode along a length of the lead 100. In the embodiment of FIG. 2, two of the electrodes 125 are ring electrodes 120. Ring electrodes typically do not enable stimulus current to be directed from only a limited angular range around a lead. Segmented electrodes 130, however, can be used to direct stimulus current to a selected angular range around a lead. When segmented electrodes are used in conjunction with an implantable pulse generator that delivers constant current stimulus, current steering can be achieved to deliver the stimulus more precisely to a position around an axis of a lead (i.e., radial positioning around the axis of a lead). To achieve current steering, segmented electrodes can be utilized in addition to, or as an alternative to, ring electrodes.

The lead 100 includes a lead body 110, terminals 135, at least one ring electrode 120, and at least one set of segmented electrodes 130 (or any other combination of electrodes). The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes 125 are made of a material that is biocompatible and does not substantially corrode under expected operating conditions in the operating environment for the expected duration of use.

Each of the electrodes 125 can either be used or unused (OFF). When an electrode is used, the electrode can be used as an anode or cathode and carry anodic or cathodic current. In some instances, an electrode might be an anode for a period of time and a cathode for a period of time.

Deep brain stimulation leads may include at least one set of segmented electrodes. Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Pat. Nos. 8,473,061; 8,571,665; 8,792,993; 9,248,272; 9,775,988; and 10,286,205; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; and 2015/0051681, all of which are incorporated herein by reference.

FIG. 3 is a schematic overview of one embodiment of components of an electrical stimulation system 300 including an electronic subassembly 310 disposed within an IPG 14 (FIG. 1). It will be understood that the electrical stimulation system can include more, fewer, or different components and can have a variety of different configurations including those configurations disclosed in the stimulator references cited herein.

The IPG 14 (FIG. 1) can include, for example, a power source 312, antenna 318, receiver 302, processor 304, and memory 303. Some of the components (for example, power source 312, antenna 318, receiver 302, processor 304, and memory 303) of the electrical stimulation system can be positioned on one or more circuit boards or similar carriers within a sealed housing of the IPG 14 (FIG. 1), if desired. Unless indicated otherwise, the term “processor” refers to both embodiments with a single processor and embodiments with multiple processors.

An external device, such as a CP or RC 306, can include a processor 307, memory 308, an antenna 317, and a user interface 319. The user interface 319 can include, but is not limited to, a display screen on which a digital user interface can be displayed and any suitable user input device, such as a keyboard, touchscreen, mouse, track ball, or the like or any combination thereof.

Any power source 312 can be used including, for example, a battery such as a primary cell battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

If the power source 312 is rechargeable battery, the battery may be recharged using the antenna 318, if desired. Power can be provided to the battery for recharging by inductively coupling the battery through the antenna to an optional recharging unit 316 external to the user. Examples of such arrangements can be found in the references identified above.

In one embodiment, electrical current is emitted by the electrodes 26 on the lead body to stimulate nerve fibers, muscle fibers, or other body tissues near the electrical stimulation system. A processor 304 is generally included to control the timing and electrical characteristics of the electrical stimulation system. For example, the processor 304 can, if desired, control one or more of the timing, frequency, amplitude, width, and waveform of the pulses. In addition, the processor 304 can select which electrodes can be used to provide stimulation, if desired. In some embodiments, the processor 304 may select which electrode(s) are cathodes and which electrode(s) are anodes. In some embodiments, the processor 304 may be used to identify which electrodes provide the most useful stimulation of the desired tissue. Instructions for the processor 304 can be stored on the memory 303. Instructions for the processor 307 can be stored on the memory 308.

Any processor 304 can be used for the IPG and can be as simple as an electronic device that, for example, produces pulses at a regular interval or the processor can be capable of receiving and interpreting instructions from the CP/RC 306 (such as CP 18 or RC 16 of FIG. 1) that, for example, allows modification of pulse characteristics. In the illustrated embodiment, the processor 304 is coupled to a receiver 302 which, in turn, is coupled to the antenna 318. This allows the processor 304 to receive instructions from an external source to, for example, direct the pulse characteristics and the selection of electrodes, if desired. Any suitable processor 307 can be used for the CP/RC 306.

Any suitable memory 303, 308 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In one embodiment, the antenna 318 is capable of receiving signals (e.g., RF signals) from an antenna 317 of a CP/RC 306 (see, CP 18 or RC 16 of FIG. 1) which is programmed or otherwise operated by a user. The signals sent to the processor 304 via the antenna 318 and receiver 302 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 300 to cease operation, to start operation, to start signal acquisition, or to stop signal acquisition. In other embodiments, the stimulation system does not include an antenna 318 or receiver 302 and the processor 304 operates as programmed.

Optionally, the electrical stimulation system 300 may include a transmitter (not shown) coupled to the processor 304 and the antenna 318 for transmitting signals back to the CP/RC 306 or another unit capable of receiving the signals. For example, the electrical stimulation system 300 may transmit signals indicating whether the electrical stimulation system 300 is operating properly or not or the level of charge remaining in the battery. The processor 304 may also be capable of transmitting information about the pulse characteristics so that a user or clinician can determine or verify the characteristics.

Transmission of signals can occur using any suitable method, technique, or platform including, but not limited to, inductive transmission, radiofrequency transmission, Bluetooth™, Wi-Fi, cellular transmission, near field transmission, infrared transmission, or the like or any combination thereof. In addition, the IPG 14 can be wirelessly coupled to the RC 16 or CP 18 using any suitable arrangement include direct transmission or transmission through a network, such as a local area network, wide area network, the Internet, or the like or any combination thereof. The CP 18 or RC 16 may also be capable of coupling to, and sending data or other information to, a network 320, such as a local area network, wide area network, the Internet, or the like or any combination thereof.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (for example, RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

A power source, such as a primary cell battery or a rechargeable battery, in the implantable control module depletes over time until the power source reaches the end of useful life or needs to be recharged. As described herein, an implantable control module, such as IPG 14, (or other device that obtains information from the implantable control module such as the RC 16 or CP 18 or any other suitable device) can estimate the capacity of the power source that has been used. In at least some embodiments, the implantable control module (or other device that obtains information from the implantable control module) can estimate a remaining capacity of the power source. It will be understood that the term “capacity” as used herein, unless indicated otherwise, can be replaced by the term “energy.” It will be understood that the terms “total capacity” or “total energy” refers to the total amount of energy or charge that can be stored on the power source. It will be understood that the terms “initial capacity” or “initial energy” refers to the total amount of energy or charge stored on the battery at an initial time (for example, at the time that the power source was created or charged or at the time that the implantable control module was implanted or manufactured.)

When the power source is a primary cell battery, the estimate of the capacity of the power source that has been used can also provide an estimate of the longevity or remaining lifetime of the primary cell battery. For example, the remaining life of the battery may be projected based on the rate of consumption of charge to that point in time (or the rate of consumption over any selected period of time). It will be understood that the term “charge” as used herein, unless indicated otherwise, can be replaced by the terms “power,” “energy,” or “capacity.”

The power source of the implantable control module provides charge for electrical stimulation as well as a variety of other operations of the implantable control module and associated elements of the electrical stimulation system. In electrical stimulation, the largest use of charge from the power source is the delivery of the electrical stimulation to the patient. The implantable control module or other device can estimate the amount of charge utilized to provide electrical stimulation for each electrical stimulation instance. In at least some instances, the electrical stimulation instance can be a particular program sequence in which the stimulation parameters, such as current amplitude, pulse width, and pulse frequency are uniform. In at least some embodiments, the electrical stimulation instance can be a particular program sequence in which the stimulation parameters vary. In at least some embodiments, the electrical stimulation may be divided into one or more time periods (e.g., timestamps) which may be uniform or nonuniform in length where each time period is considered an electrical stimulation instance.

In at least some embodiments, the implantable control module or other device can estimate the amount of charge utilized to provide electrical stimulation using one or more of the following parameters: power source voltage, current amplitude, pulse width, pulse frequency, or compliance voltage (i.e., the voltage needed to deliver the current amplitude into the tissue) for each electrode used for delivery of the electrical stimulation (i.e., each active electrode). In at least some embodiments, these parameters are stored for each stimulation instance in a database (for example, a patient usage log or the like) in the memory of the implantable control module (or stored in a memory elsewhere).

As an example, in at least some embodiments, the charge (Q) consumed to provide electrical stimulation can be estimated, for each active electrode, as the product of current amplitude (I), pulse width (PW), pulse frequency (PF), and duration (T) of the stimulation instance.

Q=I×PW×PF×T

As another example (particularly if the stimulation amplitude, pulse width, or pulse frequency is time varying), in at least some embodiments, the charge utilized to provide electrical stimulation can be estimated, for each active electrode, as the integral, over the duration of the stimulation instance, of the product of the current amplitude, pulse width, and pulse frequency.

Q=∫₀^{T}I×PW×PFdt

As yet another example, in at least some embodiments, the power (P) consumed to provide electrical stimulation can be estimated, for each active electrode, as the product of the compliance voltage (Vₕ), current amplitude (I), pulse width (PW), pulse frequency (PF), and duration (T) of the stimulation instance or as the integral, over the duration of the stimulation instance, of the product of the compliance voltage, current amplitude, pulse width, and pulse frequency.

P=Vₕ×I×PW×PF×T

P=∫₀^{T}Vₕ×I×PW×PFdt

In at least some embodiments, the compliance voltage is a predetermined value that depends on the current amplitude. In at least some embodiments, the compliance voltage is determined as a product of a tissue impedance and the current amplitude, where the tissue impedance is estimated or measured. In at least some embodiments, the tissue impedance can be estimated using a model of the tissue or using a previously measured or calculated value.

It will be recognized that any other suitable equation(s) or model can be used for determination of the charge or power consumed to provide electrical stimulation.

The determined charge or power for each electrical stimulation instance can be summed to determine the total amount of charge or power, as well as to determine the capacity of the power source, used for electrical stimulation.

In at least some embodiments, the delivery of electrical stimulation is the only use of charge that is considered in the determination of capacity used from the power source. In at least some of these embodiments, this total amount of charge can be subtracted from the total storage capacity or total storage energy (or estimated total capacity) of the power source to estimate the amount of capacity remaining in the power source.

There are other operations of the implantable control module that consume charge from the power source. In at least some embodiments, the determination of the estimate of a capacity of the power source that has been used includes accounting for one or more of these operations. Examples of operations include, but are not limited to, operation of the processor; operation of digital timers; operation of a step-up converter; operation of current sources; operation of reference sources; operation of a memory (e.g., reading or writing); or operation of communications components (such as the antenna 318 or receiver 302 of FIG. 3) of the implantable pulse generator. Examples of implantable control modules and components that consume charge from the power source include, but are not limited to, U.S. Pat. Nos. 6,181,969; 6,516,227; 6,609,029; 6,609,032; 6,741,892; 6,895,280; 7,949,395; 7,244,150; 7,672,734; and 7,761,165; 7,974,706; 8,175,710; 8,224,450; and 8,364,278; and U.S. Patent Application Publication No. 2007/0150036, all of which are incorporated herein by reference in their entireties.

A variety of different methods can be utilized for estimating the charge consumed by these operations. Moreover, different methods for estimation can be used for different operations or sets of operations.

For example, in at least some embodiments, an overhead consumption value can be provided to account for one or more operations (which may or may not be specified). In at least some embodiments, the overhead consumption value is preselected and may be defined, for example, as a predetermined amount of charge for a given period of time. In at least some embodiments, the overhead consumption value may be different for different modes of the implantable control module or electrical stimulation system. For example, the overhead consumption value may be different for a period of no stimulation than for periods of stimulation. In at least some embodiments, the overhead consumption value may be different for different stimulation programs.

As a second example, for one or more of the operation(s), the estimated charge consumption can be calculated or estimated for each instance or time period of an operation (or set of two or more operations). In at least some embodiments, the calculation or estimation can be performed using any suitable variables, such as voltage, current, impedance, or the like.

As a third example, for one or more of the operation(s), the charge consumption can be estimated using an average charge consumption for each operation (or set of two or more operations) over a given period of time. In at least some embodiments, the estimation can use predetermined values for the operation(s). In at least some embodiments, the estimation may be different for different modes of the implantable control module or electrical stimulation system.

Any combination of these methods can be used to account for charge consumed by two or more operations. For example, one or more first operations can be accounted for using the overhead consumption value, one or more second operations can be account for by calculating or estimating the consumption for each instance, and one or more third operations can be accounted for using an average charge consumption for the operation(s). In at least some embodiments, the method of accounting for a particular operation can vary depending on the mode of the implantable control module or electrical stimulation system or for any other reason.

The amount of charge consumed for the operations can be combined for different time periods to obtain a total. Charge consumption for these additional operations can be combined with the charge consumption for the electrical stimulation to obtain a total amount of charge consumed.

In at least some embodiments, the processor 304 of the implantable control module, such as IPG 14, can determine the charge consumption, the estimate of the capacity of the power source that has been used, or any combination thereof. In other embodiments, the RC 16, CP 18, or another device can retrieve information as described above (for example, data or usage logs) from the memory 303 of the implantable control module, such as IPG 14, and then determine the charge consumption, the estimate of the capacity of the power source that has been used, or any combination thereof. In at least some embodiments, the information (for example, data or usage logs) from the memory 303 of the implantable control module can be uploaded to the RC 16, CP 18, other device, or the cloud or other data storage arrangement for use in determining the charge consumption, the estimate of the capacity of the power source that has been used, or any combination thereof.

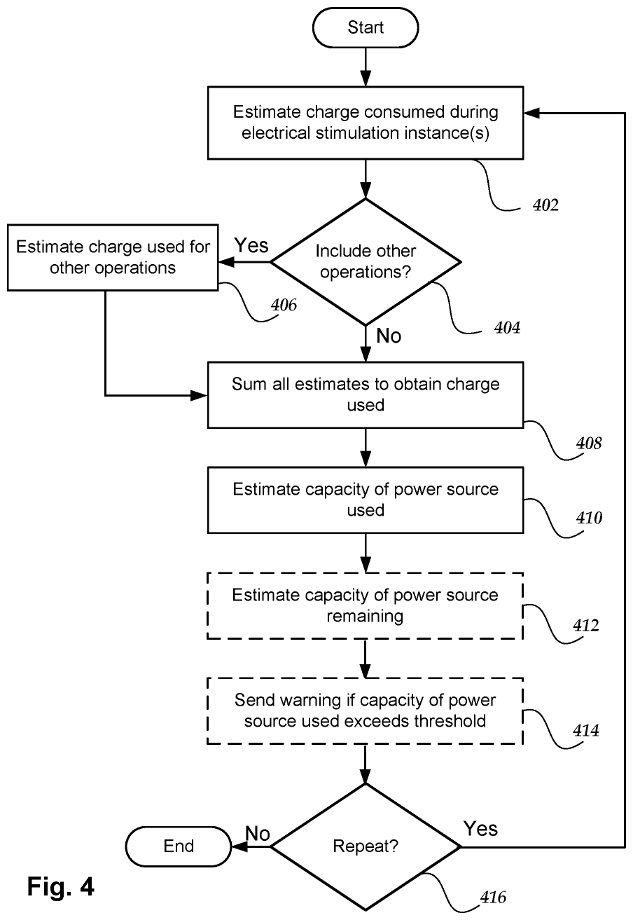

FIG. 4 illustrates one embodiment of a method for estimating a capacity of a power source that has been used. In step 402, the charge consumed during one or more electrical stimulation instances is determined, as described above.

In step 404, a query is made whether the consumption of charge by other operations is to be included. If yes, then in step 406 the charge consumption for those other operations is estimated or otherwise determined. For example, any of the methods or combinations of the methods described above for estimating the charge consumption for these operations can be used. In at least some embodiments, steps 404 and 406 can be deleted.

In step 408, all of the estimates are combined to estimate the amount of charge that has been used or consumed. In step 410, the capacity of the power source that has been used is estimated from amount of charge that has been used or consumed. In optional step 412, the remaining capacity of the power source can be estimated based on the capacity of the power source that has been used.

In optional step 414, a warning can be sent to the patient or a device, such as the RC 16, CP 18, or other device (for example, the computer of a clinician or other caregiver), when the capacity of the power source that has been used exceeds a threshold amount. For example, his warning can indicate that the power source should be replaced or recharged.

In step 416, the method can return to step 402 for additional stimulation instances or time periods.

In at least some embodiments, the determination can be used to monitor the longevity of a power source in the implantable control module. In at least some embodiments, the ability to make real-time longevity estimates can help guide selection or use of different programming options for the electrical stimulation. In at least some embodiment, the determination can provide a more accurate replacement warning or a longevity indicator.

In at least some embodiments, the determination of the estimate of the capacity of the power source that has been used can alert a clinician, programmer, or patient of high usage programs or program changes. In at least some embodiments, the estimates of the capacity of the power source that has been used can facilitate identifying programming models that reduce power source usage.

It will be understood that each block of the flowcharts, and combinations of blocks in the flowcharts and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system, comprising:
a lead comprising a plurality of electrodes disposed along a distal portion of the lead; and
an implantable control module coupled, or coupleable, to the lead and configured for implantation in a patient, the implantable control module comprising
a power source, and
a processor coupled to the power source and configured for directing electrical stimulation through the electrodes of the lead using the power source and for calculating an estimate of a capacity or energy of the power source that has been used based, at least in part, on the directed electrical stimulation.

2. The electrical stimulation system of claim 1, wherein the estimate of the capacity or energy of the power source that has been used comprises an estimate of the charge or power used during each electrical stimulation instance in which the processor directs the electrical stimulation through the electrodes of the lead.

3. The electrical stimulation system of claim 2, wherein the estimate of the charge used during an electrical stimulation instance comprises a summation of, for each of the electrodes used for delivery of the electrical stimulation during the electrical stimulation instance, a) a product of a stimulation amplitude, pulse width, pulse frequency, and duration of the electrical stimulation instance for the electrode orb) a product of the stimulation amplitude, pulse width, and pulse frequency integrated over the duration of the electrical stimulation instance for the electrode.

4. The electrical stimulation system of claim 2, wherein the estimate of the power used during an electrical stimulation instance comprises a summation of, for each of the electrodes used for delivery of the electrical stimulation during the electrical stimulation instance, a) a product of a compliance voltage, stimulation amplitude, pulse width, pulse frequency, and duration of the electrical stimulation instance for the electrode orb) a product of the compliance voltage, stimulation amplitude, pulse width, and pulse frequency integrated over the duration of the electrical stimulation instance for the electrode.

5. The electrical stimulation system of claim 3, wherein the compliance voltage is determined using an estimate or measurement of tissue impedance and the stimulation amplitude.

6. The electrical stimulation system of claim 1, wherein the estimate of the capacity or energy of the power source that has been used is also based on an estimate of charge or power used for one or more other operations of the implantable pulse generator.

7. The electrical stimulation system of claim 6, wherein the estimate of the charge or power used for the one or more other operations of the implantable pulse generator comprises at least one calculation or estimation of the charge or power used for at least one instance of at least one of the one or more other operations.

8. The electrical stimulation system of claim 6, wherein the estimate of the charge or power used for the one or more operations of the implantable pulse generator uses an overhead consumption value for accounting for at least one of the one or more other operations.

9. The electrical stimulation system of claim 6, wherein the estimate of the charge or power used for the one or more operations of the implantable pulse generator uses an average charge consumption over a predefined period of time for at least one of the one or more other operations.

10. The electrical stimulation system of claim 6, wherein the one or more other operations of the implantable pulse generator comprise at least one of the following: operation of the processor; operation of digital timers; operation of a step-up converter; operation of current sources; operation of reference sources; operation of a memory; or operation of communications components of the implantable pulse generator.

11. A method for estimating a capacity or energy of a power source of an implantable control module of an electrical stimulation system that has been used during electrical stimulation, the method comprising:
estimating a charge or power used during each of a plurality of electrical stimulation instances;
combining the estimates of the charge or power used during each of the electrical stimulation instances;
determining the capacity or energy of the power source that has been used utilizing the combined estimates of the charge or power used during each of the electrical stimulation instances; and
providing a warning to a patient or device when the determined capacity or energy exceeds a threshold value.

12. The method of claim 11, wherein estimating the charge used during an electrical stimulation instance comprises determining a summation of, for each of the electrodes used for delivery of the electrical stimulation during the electrical stimulation instance, a) a product of a stimulation amplitude, pulse width, pulse frequency, and duration of the electrical stimulation instance for the electrode orb) a product of the stimulation amplitude, pulse width, and pulse frequency integrated over the duration of the electrical stimulation instance for the electrode.

13. The method of claim 11, wherein estimating the power used during an electrical stimulation instance comprises determining a summation of, for each of the electrodes used for delivery of the electrical stimulation during the electrical stimulation instance, a) a product of a compliance voltage, stimulation amplitude, pulse width, pulse frequency, and duration of the electrical stimulation instance for the electrode orb) a product of the compliance voltage, stimulation amplitude, pulse width, and pulse frequency integrated over the duration of the electrical stimulation instance for the electrode.

14. The method of claim 13, further comprising determining the compliance voltage using an estimate or measurement of tissue impedance and the stimulation amplitude.

15. The method of claim 11, further comprising:
estimating a charge or power used for one or more other operations of the implantable pulse generator; and
combining the estimates of the charge or power used for the one or more other operations,
wherein determining the capacity or energy of the power source that has been used comprises determining the capacity or energy of the power source that has been used utilizing the combined estimates of the charge or power used during each of the electrical stimulation instances and the combined estimate of the charge or power used for the one or more other operations.

16. The method of claim 15, wherein estimating the charge or power used for the one or more other operations of the implantable pulse generator comprises calculating or estimating the charge or power used for at least one instance of at least one of the one or more other operations.

17. The method of claim 15, wherein estimating the charge or power used for the one or more operations of the implantable pulse generator comprises using an overhead consumption value for accounting for at least one of the one or more other operations.

18. The method of claim 15, wherein estimating the charge or power used for the one or more operations of the implantable pulse generator comprises using an average charge consumption over a predefined period of time for at least one of the one or more other operations.

19. The method of claim 15, wherein the one or more other operations of the implantable pulse generator comprise at least one of the following: operation of the processor; operation of digital timers; operation of a step-up converter; operation of current sources; operation of reference sources; operation of a memory; or operation of communications components of the implantable pulse generator.

20. The method of claim 11, further comprising delivering electrical stimulation to a patient for each of the electrical stimulation instances.
